(19) United States
(12) Patent Application Publication
(10) Pub. No.: US 2002/0194625 A1
(43) Pub. Date: Dec. 19, 2002

(54) Transgenic animals expressing heparanase and its uses

(75) Inventors: Eyal Zcharia, Kiryat Hayovel (IL); Israel Vlodavsky, Mevaseret Zion (IL); Shula Metzger, Beit Hakerem (IL); Tova Chajek-Shaul, Ramat Sharett (IL); Orit Goldshmidt, Kiryat Yovel (IL); Iris Pecker, Rishon LeZion (IL); Neta Ilan, Rehovot (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Insight Strategy & Marketing Ltd.

(21) Appl. No.: 09/864,321

(22) Filed: May 25, 2001

(51) Int. Cl. A01K 67/027; C12N 9/24

(52) U.S. Cl. 800/7; 800/14; 800/19; 435/200

(57) ABSTRACT

A transgenic animal expressing heparanase from a transgene, methods for its preparation, compositions-of-matter derived therefrom and its uses.

### FIELD AND BACKGROUND OF THE INVENTION

The present invention relates to transgenic animals expressing heparanase and to the uses thereof as a model for human disease and for the commercial production of heparanase.

Glycos(Iminoglycans (GA Gs):

GAGs are polymers of repeated disaccharide units consisting of uronic acid and a hexosamine. Biosynthesis of GAGs except hyaluronic acid is initiated from a core protein. Proteoglycans may contain several GAG side chains from similar or different families. GAGs are synthesized as homopolymers which may subsequently be modified by N-deacetylation and N-sulfation, followed by C5-epimerization of glucuronic acid to iduronic acid and O-sulfation. The chemical composition of GAGs from various tissues varies to a great extent.

The natural metabolism of GAGs in animals is carried out by hydrolysis. Generally, the GAGs are degraded in a two step procedure. First the proteoglycans are internalized in endosomes, where initial depolymerization of the GAG chain takes place. This step is mainly hydrolytic and yields oligosaccharides. Further degradation is carried out following fusion with lysosome, where desulfation and exolytic depolymerization to monosaccharides take place (42).

The only GAG degrading endolytic enzymes characterized so far in animals are the hyaluronidases. The hyaluronidases are a family of 1-4 endoglucosaminidases that depolymerize hyaluronic acid and chondroitin sulfate. The cDNAs encoding sperm associated PH-20 (Hyal3), and the lysosomal hyaluronidases Hyal 1 and Hyal 2 were cloned and published (27). These enzymes share an overall homology of 40% and have different tissue specificities, cellular localizations and pH optima for activity.

Exolytic hydrolases are better characterized, among which are beta-glucuronidase, alpha-L-iduronidase and beta-N-acetylglucosaminidase. In addition to hydrolysis of the glycosidic bond of the polysaccharide chain, GAG degradation involves desulfation, which is catalyzed by several lysosomal sulfatases such as N-acetylgalactosamine-4-sulfatase, iduronate-2-sulfatase and heparin sulfamidase. Deficiency in any of lysosomal GAG degrading enzymes results in a lysosomal storage disease known as mucopolysaccharidosis.

Glycosyl Hydrolases:

Glycosyl hydrolases are a widespread group of enzymes that hydrolyze the o-glycosidic bond between two or more carbohydrates or between a carbohydrate and a noncarbohydrate moiety. The enzymatic hydrolysis of glycosidic bond occurs by one or two major mechanisms leading to overall retention or inversion of the anomeric configuration. In both mechanisms, catalysis involves a proton donor and a nucleophile. Glycosyl hydrolyses have been classified into 58 families based on amino acid similarities. The glycosyl hydrolyses from families 1, 2, 5, 10, 17, 30, 35, 39 and 42 act on a large variety of substrates, however, they all hydrolyze the glycosidic bond in a general acid catalysis mechanism, with retention of the anomeric configuration. The mechanism involves two glutamic acid residues, which serve as the proton donor and the nucleophile, with an asparagine, which always precedes the proton donor. Analyses of a set of known 3D structures from this group of enzymes revealed that their catalytic domains, despite the low level of sequence identity, adopt a similar (alpha/beta) 8 fold with the proton donor and the nucleophile located at the C-terminal ends of strands beta 4 and beta 7, respectively. Mutations in the functional conserved amino acids of lysosomal glycosyl hydrolases were identified in lysosomal storage diseases.

Lysosomal glycosyl hydrolases including beta-glucuronidase, beta-manosidase, beta-glucocerebrosidase, beta-galactosidase and alpha-L-iduronidase, are all exo-glycosyl hydrolases, belong to the GH-A clan and share a similar catalytic site. However, many endo-glucanases from various organisms, such as bacterial and fungal xylenases and cellulases share this catalytic domain (1).

Heparan Sulfate Proteoglycans (HSPGs):

HSPGs are ubiquitous macromolecules associated with the cell surface and extracellular matrix (ECM) of a wide range of cells of vertebrate and invertebrate tissues (3-7). The basic HSPG structure consists of a protein core to which several linear heparan sulfate chains are covalently attached. The polysaccharide chains are typically composed of repeating hexuronic and D-glucosamine disaccharide units that are substituted to a varying extent with N- and O-linked sulfate moieties and N-linked acetyl groups (3-7). Studies on the involvement of ECM molecules in cell attachment, growth and differentiation revealed a central role of HSPGs in embryonic morphogenesis, angiogenesis, metastasis, neurite outgrowth and tissue repair (3-7). The heparan sulfate (HS) chains, which are unique in their ability to bind a multitude of proteins, ensure that a wide variety of effector molecules cling to the cell surface (6-8). HSPGs are also prominent components of blood vessels (5). In large vessels they are concentrated mostly in the intima and inner media, whereas in capillaries they are found mainly in the subendothelial basement membrane where they support proliferating and migrating endothelial cells and stabilize the structure of the capillary wall. The ability of HSPGs to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion. Cleavage of HS may therefore result in disassembly of the subendothelial ECM and hence may play a decisive role in extravasation of normal and malignant blood-borne cells (9-11). HS catabolism is observed in inflammation, wound repair, diabetes, and cancer metastasis, suggesting that enzymes, which degrade HS, play important roles in pathologic processes.

Heparanase:

Heparanase is a glycosylated enzyme that is involved in the catabolism of certain glycosaminoglycans. It is an endoglucuronidase that cleaves heparan sulfate at specific intrachain sites (12-15). Interaction of T and B lymphocytes, platelets, granulocytes, macrophages and mast cells with the subendothelial extracellular matrix (ECM) is associated with degradation of heparan sulfate by heparanase activity (16). Placental heparanase acts as an adhesion molecule or as a degradative enzyme depending on the pH of the microenvironment (17).

Heparanase is released from intracellular compartments (e.g., lysosomes, specific granules) in response to various activation signals (e.g., thrombin, calcium ionophores, immune complexes, antigens and mitogens), suggesting its regulated involvement in inflammation and cellular immunity responses (16).

It was also demonstrated that heparanase can be readily released from human neutrophils by 60 minutes incubation at 4° C. in the absence of added stimuli (18).

Gelatinase, another ECM degrading enzyme, which is found in tertiary granules of human neutrophils with heparanase, is secreted from the neutrophils in response to phorbol 12-myristate 13-acetate (PMA) treatment (19-20).

In contrast, various tumor cells appear to express and secrete heparanase in a constitutive manner in correlation with their metastatic potential (21).

Degradation of heparan sulfate by heparanase results in the release of heparin-binding growth factors, enzymes and plasma proteins that are sequestered by heparan sulfate in basement membranes, extracellular matrices and cell surfaces (22-23).

Heparanase activity has been described in a number of cell types including cultured skin fibroblasts, human neutrophils, activated rat T-lymphocytes, normal and neoplastic murine B-lymphocytes, human monocytes and human umbilical vein endothelial cells, SK hepatoma cells, human placenta and human platelets.

Procedures for purification of natural heparanase were reported for SK hepatoma cells and human placenta (U.S. Pat. No. 5,362,641) and for human platelets derived enzymes (53).

Cloning and Expression of the Human Heparanase Gene (hpa):

The human hpa cDNA, which encodes human heparanase, was cloned from human placenta. It contains an open reading frame, which encodes a polypeptide of 543 amino acids with a calculated molecular weight of 61,192 daltons (2). The cloning procedures of the hpa cDNA and genomic DNA from several species are described in length in U.S. Pat. No. 5,986,822, U.S. patent application Ser. Nos. 09/109,386 and 09/258,892 and PCT Application No. US98/17954, all of which are incorporated herein by reference. An identical cDNA encoding human heparanase was isolated later on from hepatoma cell line SK-hep1 (54). From platelets (55, 57, PCT/US99/0 1489, PCT/AU98/00898) and from SV40 transformed fibroblasts (56, PCT/EP99/00777).

The genomic locus, which encodes heparanase, spans about 40 kb. It is composed of 12 exons separated by 11 introns and is localized on human chromosome 4.

The ability of the hpa gene product to catalyze degradation of heparan sulfate (HS) in vitro was examined by expressing the entire open reading frame of hpa in High five and Sf21 insect cells, and the mammalian human 293 embryonic kidney cell line expression systems. Extracts of infected or transfected cells were assayed for heparanase catalytic activity. For this purpose, cell lysates were incubated with sulfate labeled, ECM-derived HSPG (peak I), followed by gel filtration analysis (Sepharose 6B) of the reaction mixture. While the substrate alone consisted of high molecular weight material, incubation of the HSPG substrate with lysates of cells infected or transfected with hpa containing vectors resulted in a complete conversion of the high molecular weight substrate into low molecular weight labeled heparan sulfate degradation fragments (see, for example, U.S. Pat. application Ser. No. 09/071,618, which is incorporated herein by reference.

In other experiments, it was demonstrated that the heparanase enzyme expressed by cells infected with a pFhpa virus is capable of degrading HS complexed to other macromolecular constituents (e.g., fibronectin, laminin, collagen) present in a naturally produced intact ECM (see U.S. Pat. application Ser. No. 09/109,386, which is incorporated herein by reference), in a manner similar to that reported for highly metastatic tumor cells or activated cells of the immune system (7, 8).

In human primary fibroblasts transfected with the heparanase cDNA the enzyme was localized to the lysosomes.

Preferential Expression of the hpa Gene in Human Breast and Hepatocellular Carcinomas:

Semi-quantitative RT-PCR was employed to evaluate the expression of the hpa gene by human breast carcinoma cell lines exhibiting different degrees of metastasis. A marked increase in hpa gene expression is observed, which correlates to metastatic capacity of non-metastatic MCF-7 breast carcinoma, moderately metastatic MDA 231 and highly metastatic MDA 435 breast carcinoma cell lines. Significantly, the differential pattern of the hpa gene expression correlated with the pattern of heparanase activity.

Expression of the hpa gene in human breast carcinoma was demonstrated by in situ hybridization to archival paraffin embedded human breast tissue. Hybridization of the heparanase antisense riboprobe to invasive duct carcinoma tissue sections resulted in a massive positive staining localized specifically to the carcinoma cells. The hpa gene was also expressed in areas adjacent to the carcinoma showing fibrocystic changes. Normal breast tissue derived from reduction mammoplasty failed to express the hpa transcript. High expression of the hpa gene was also observed in tissue sections derived from human hepatocellular carcinoma specimens but not in normal adult liver tissue. Furthermore, tissue specimens derived from adenocarcinoma of the ovary, squamous cell carcinoma of the cervix and colon adenocarcinoma exhibited strong staining with the hpa RNA probe, as compared to a very low staining of the hpa mRNA in the respective non-malignant control tissues (2).

A preferential expression of heparanase in human tumors versus the corresponding normal tissues was also noted by immunohistochemical staining of paraffin embedded sections with monoclonal anti-heparanase antibodies. Positive cytoplasmic staining was found in neoplastic cells of the colon carcinoma and in dysplastic epithelial cells of a tubulovillous adenoma found in the same specimen while there was little or no staining of the normal looking colon epithelium located away from the carcinoma. Of particular significance was an intense immunostaining of colon adenocarcinoma cells that had metastasized into lymph node, lung and liver, as compared to the surrounding normal tissues (58).

Latent and Active Forms of the Heparanase Protein:

The apparent molecular size of the recombinant enzyme produced in the baculovirus expression system was about 65 kDa. This heparanase polypeptide contains 6 potential N-glycosylation sites. Following deglycosylation by treatment with peptide N-glycosidase, the protein appeared as a 57 kDa band. This molecular weight corresponds to the deduced molecular mass (61,192 daltons) of the 543 amino acid polypeptide encoded by the full length hpa eDNA after cleavage of the predicted 3 kDa signal peptide. No further reduction in the apparent size of the N-deglycosylated protein was observed following concurrent O-glycosidase and neuraminidase treatment. Deglycosylation had no detectable effect on enzymatic activity.

Unlike the baculovirus enzyme, expression of the full length heparanase polypeptide in mammalian cells (e.g., 293 kidney cells, CHO) yielded a major protein of about 50 kDa and a minor of about 65 kDa in cell lysates. Comparison of the enzymatic activity of the two forms, using a semi-quantitative gel filtration assay, revealed that the 50 kDa enzyme is at least 100-200 fold more active than the 65 kDa form. A similar difference was observed when the specific activity of the recombinant 65 kDa baculovirus enzyme was compared to that of the 50 kDa heparanase preparations purified from human platelets, SK-hep-1 cells, or placenta. These results suggest that the 50 kDa protein is a mature processed form of a latent heparanase precursor. Amino terminal sequencing of the platelet heparanase indicated that cleavage occurs between amino acids Gln¹⁵⁷ and Lys¹⁵⁸. As indicated by the hydropathic plot of heparanase, this site is located within a hydrophillic peak, which is likely to be exposed and hence accessible to proteases.

According to Fairbank et al. (57) the precursor is cleaved at three sites to form a heterodimer of a 50 kDa polypeptide (the mature form) that is associated with a 8 kDa peptide.

Involvement of Heparanase in Tumor Cell Invasion and Metastasis:

Circulating tumor cells arrested in the capillary beds often attach at or near the intercellular junctions between adjacent endothelial cells. Such attachment of the metastatic cells is followed by rupture of the junctions, retraction of the endothelial cell borders and migration through the breach in the endothelium toward the exposed underlying base membrane (BM) (24). Once located between endothelial cells and the BM, the invading cells must degrade the subendothelial glycoproteins and proteoglycans of the BM in order to migrate out of the vascular compartment. Several cellular enzymes (e.g., collagenase IV, plasminogen activator, cathepsin B, elastase, etc.) are thought to be involved in degradation of BM (25). Among these enzymes is heparanase that cleaves HS at specific intrachain sites (16, 11). Expression of a HS degrading heparanase was found to correlate with the metastatic potential of mouse lymphoma (26), fibrosarcoma and melanoma (21) cells. Moreover, elevated levels of heparanase were detected in sera from metastatic tumor bearing animals and melanoma patients (21) and in tumor biopsies of cancer patients (12).

The inhibitory effect of various non-anticoagulant species of heparin on heparanase was examined in view of their potential use in preventing extravasation of blood-borne cells. Treatment of experimental animals with heparanase inhibitors markedly reduced (>90%) the incidence of lung metastases induced by B 16 melanoma, Lewis lung carcinoma and mammary adenocarcinoma cells (12, 13, 28). Heparin fractions with high and low affinity to anti-thrombin III exhibited a comparable high anti-metastatic activity, indicating that the heparanase inhibiting activity of heparin, rather than its anticoagulant activity, plays a role in the anti-metastatic properties of the polysaccharide (12).

The direct role of heparanase in cancer metastasis was demonstrated by two experimental systems. The murine T-lymphoma cell line Eb has no detectable heparanase activity. Whether the introduction of the hpa gene into Eb cells would confer a metastatic behavior on these cells was investigated. To this purpose, Eb cells were transfected with a full length human hpa cDNA. Stable transfected cells showed high expression of the heparanase mRNA and enzyme activity. These hpa and mock transfected Eb cells were injected subcutaneously into DBA/2 mice and mice were tested for survival time and liver metastases. All mice (n=20) injected with mock transfected cells survived during the first 4 weeks of the experiment, while 50% mortality was observed in mice inoculated with Eb cells transfected with the hpa cDNA. The liver of mice inoculated with hpa transfected cells was infiltrated with numerous Eb lymphoma cells, as was evident both by macroscopic evaluation of the liver surface and microscopic examination of tissue sections. In contrast, metastatic lesions could not be detected by gross examination of the liver of mice inoculated with mock transfected control Eb cells. Few or no lymphoma cells were found to infiltrate the liver tissue. In a different model of tumor metastasis, transient transfection of the heparanase gene into low metastatic B16-Fl mouse melanoma cells followed by intravenous inoculation, resulted in a 4- to 5-fold increase in lung metastases.

Finally, heparanase externally adhered to B16-F1 melanoma cells increased the level of lung metastases in C57BL mice as compared to control mice (see U.S. Pat. application Ser. No. 09/260,037, which is incorporated herein by reference).

Possible Involvement of Heparanase in Tumor Angiogenesis:

Fibroblast growth factors are a family of structurally related polypeptides characterized by high affinity to heparin (29). They are highly mitogenic for vascular endothelial cells and are among the most potent inducers of neovascularization (29-30). Basic fibroblast growth factor (bFGF) has been extracted from a subendothelial ECM produced in vitro (31) and from basement membranes of the cornea (32), suggesting that to ECM may serve as a reservoir for bFGF. Immunohistochemical staining revealed the localization of bFGF in basement membranes of diverse tissues and blood vessels (23). Despite the ubiquitous presence of bFGF in normal tissues, endothelial cell proliferation in these tissues is usually very low, suggesting that bFGF is somehow sequestered from its site of action. Studies on the interaction of bFGF with ECM revealed that bFGF binds to HSPG in the ECM and can be released in an active form by HS degrading enzymes (33, 32, 34). It was demonstrated that heparanase activity expressed by platelets, mast cells, neutrophils, and lymphoma cells is involved in release of active bFGF from ECM and basement membranes (35), suggesting that heparanase activity may not only function in cell migration and invasion, but may also elicit an indirect neovascular response. These results suggest that the ECM HSPG provides a natural storage depot for bFGF and possibly other heparin-binding growth promoting factors (36, 37). Displacement of bFGF from its storage within basement membranes and ECM may therefore provide a novel mechanism for induction of neovascularization in normal and pathological situations.

Recent studies indicate that heparin and HS are involved in binding of bFGF to high affinity cell surface receptors and in bFGF cell signaling (38, 39). Moreover, the size of HS required for optimal effect was similar to that of HS fragments released by heparanase (40). Similar results were obtained with vascular endothelial cells growth factor (VEGF) (41), suggesting the operation of a dual receptor mechanism involving HS in cell interaction with heparin-binding growth factors. It is therefore proposed that restriction of endothelial cell growth factors in ECM prevents their systemic action on the vascular endothelium, thus maintaining a very low rate of endothelial cells turnover and vessel growth. On the other hand, release of bFGF from storage in ECM as a complex with HS fragment, may elicit localized endothelial cell proliferation and neovascularization in processes such as wound healing, inflammation and tumor development (36, 37).

The Involvement of Heparanase in other Physiological Processes and its Potential Therapeutic Applications:

Apart from its involvement in tumor cell metastasis, inflammation and autoimmunity, mammalian heparanase may be applied to modulate bioavailability of heparin-binding growth factors; cellular responses to heparin-binding growth factors (e.g., bFGF, VEGF) and cytokines (IL-8) (44, 41); cell interaction with plasma lipoproteins (49); cellular susceptibility to certain viral and some bacterial and protozoa infections (45-47); and disintegration of amyloid plaques (48).

Viral Infection:

The presence of heparan sulfate on cell surfaces have been shown to be the principal requirement for the binding of Herpes Simplex (45) and Dengue (46) viruses to cells and for subsequent infection of the cells. Removal of the cell surface heparan sulfate by heparanase may therefore abolish virus infection. In fact, treatment of cells with bacterial heparitinase (degrading heparan sulfate) or heparinase (degrading heparan) reduced the binding of two related animal herpes viruses to cells and rendered the cells at least partially resistant to virus infection (45). There are some indications that the cell surface heparan sulfate is also involved in HIV infection (47).

Neurodegenerative Diseases:

Heparan sulfate proteoglycans were identified in the prion protein amyloid plaques of Genstmann-Straussler Syndrome, Creutzfeldt-Jakob disease and Scrape (48). Heparanase may disintegrate these amyloid plaques, which are also thought to play a role in the pathogenesis of Alzheimer's disease.

Restenosis and atherosclerosis: Proliferation of arterial smooth muscle cells (SMCs) in response to endothelial injury and accumulation of cholesterol rich lipoproteins are basic events in the pathogenesis of atherosclerosis and restenosis (50). Apart from its involvement in SMC proliferation as a low affinity receptor for heparin-binding growth factors, HS is also involved in lipoprotein binding, retention and uptake (51). It was demonstrated that HSPG and lipoprotein lipase participate in a novel catabolic pathway that may allow substantial cellular and interstitial accumulation of cholesterol rich lipoproteins (49). The latter pathway is expected to be highly atherogenic by promoting accumulation of apoB and apoE rich lipoproteins (e.g., LDL, VLDL, chylomicrons), independent of feed back inhibition by the cellular cholesterol content. Removal of SMC HS by heparanase is therefore expected to inhibit both SMC proliferation and lipid accumulation and thus may halt the progression of restenosis and atherosclerosis.

Pulmonary Diseases:

The data obtained from the literature suggests a possible role for GAGs degrading enzymes, such as, but not limited to, heparanases, connective tissue activating peptide, heparinases, hyluronidases, sulfatases and chondroitinases, in reducing the viscosity of sinuses and airway secretions with associated implications on curtailing the rate of infection and inflammation. The sputum from CF patients contains at least 3% GAGs, thus contributing to its volume and viscous properties. It was shown that heparanase reduces the viscosity of sputum of Cystic fibrosis (CF) patients (see, U.S. patent application Ser. No. 09/046,475). Recombinant heparanase has been shown to reduce viscosity of sputum of CF patients (see, U.S. Pat. application Ser. No. 09/046,475).

Heparanase and/or heparanase inhibitors may thus prove useful for treating conditions such as wound healing, angiogenesis, restenosis, atherosclerosis, inflammation, neurodegenerative diseases and viral infections. Mammalian heparanase can be used to neutralize plasma heparin, as a potential replacement of protamine.

There is, thus, a widely recognized need for, and it would be highly advantageous to have, transgenic animals producing heparanase so as to efficiently produce commercial quantities of this enzyme. Such transgenic animals may also find uses as models for human disease associated with impaired heparanase expression, such as, for example, metastasis.

### SUMMARY OF THE INVENTION

According to one aspect of the present invention there is provided a transgenic animal expressing heparanase from a transgene.

According to further features in preferred embodiments of the invention described below, the transgenic animal is homozygous for the transgene.

According to still further features in the described preferred embodiments the transgenic animal is heterozygous for the transgene.

According to still further features in the described preferred embodiments the transgenic animal has a single locus harboring the transgene.

According to still further features in the described preferred embodiments the transgenic animal has at least two loci each harboring the transgene.

According to still further features in the described preferred embodiments the heparanase is human heparanase.

According to still further features in the described preferred embodiments the human heparanase is genetically modified to be cleavable into an active form via a protease.

According to still further features in the described preferred embodiments the heparanase is processed by an endogenous protease of the animal into an active form.

According to still further features in the described preferred embodiments the transgene encodes a processed and active form of heparanase.

According to still further features in the described preferred embodiments the heparanase is expressed under control of a tissue specific promoter.

According to still further features in the described preferred embodiments the heparanase is expressed under control of a tissue non-specific promoter.

According to still further features in the described preferred embodiments the heparanase is expressed under control of a constitutive promoter.

According to still further features in the described preferred embodiments the heparanase is expressed under control of an inducible promoter.

According to still further features in the described preferred embodiments the transgenic animal is a mammal.

According to still further features in the described preferred embodiments the heparanase being expressed in and secreted by cells of a mammary glands of the mammal.

Thus, according to another aspect of the present invention there is provided a method of manufacturing heparanase, the method comprising the steps of (a) obtaining a transgenic mammal having mammary glands, the mammal expressing recombinant heparanase and secreting the heparanase into milk being produced by the mammary glands; (b) milking the mammal so as to obtain milk containing heparanase; and (c) purifying the heparanase from the milk.

Hence, according to another aspect of the present invention there is provided a composition of matter comprising milk containing heparanase.

According to still further features in the described preferred embodiments the transgenic animal is an avian.

According to still further features in the described preferred embodiments the heparanase being expressed in and secreted by egg producing cells of the avian.

Thus, according to yet another aspect of the present invention there is provided a method of manufacturing heparanase, the method comprising the steps of (a) obtaining a transgenic avian having egg producing cells, the mammal expressing recombinant heparanase and secreting the heparanase into eggs being produced by the egg producing cells; (b) collecting eggs laid by the avian so as to obtain eggs containing heparanase; and (c) purifying the heparanase from the eggs.

Hence, according to yet another aspect of the present invention there is provided a composition of matter comprising egg containing heparanase.

According to still additional aspects of the invention, there are provided sex cells (sperm cells or oocytes) and embryos derived from a transgenic animal of the invention.

The present invention successfully addresses the shortcomings of the presently known configurations by providing transgenic animals expressing heparanase which can be used as animal models and/or for commercial production of recombinant heparanase.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-Div demonstrate the expression of the heparanase protein in various tissues of homozygous transgenic mice overexpressing the human hpa gene. 1A—Western blot analysis; 1Bi-iii—Heparanase activity (wild=wild type control mice; transg.=transgenic mice); 1Ci-iv Immunohistochemistry of colon and heart tissues (1Ci and 1Ciii-transgenic mice, 1Cii and 1Civ—control mice). Western analysis and immunohistochemistry were performed using the anti heparanase monoclonal antibody HP-130.

FIGS. 2A-D show morphological appearance of mammary glands (whole mount) from control (2A and 2C) vs. transgenic (2B and 2D) mice overexpressing the hpa gene in all tissues.

FIG. 3 demonstrates binding of bFGF to embryonic fibroblasts. Fibroblasts isolated from 15 days embryos of heparanase transgenic (Tg/Hep) and control mice were incubated with various concentrations of ¹²⁵I-b-FGF. Following incubation cells were washed and the bound b-FGF was quantitated.

FIG. 4 demonstrates heparanase activity in milk of transgenic mice. Milk samples from two independent lines of heparanase transgenic mice, G1 and G3, and from control mice were incubated with 35S labeled ECM for 48 hours. Following incubation degradation products were size fractionated. Heparanase activity is detected in the milk of G3 and G1 transgenic mice and not in control mice.

### DESCRIPTION OF THE PREFERRED EMBODIMENTS

The present invention is of transgenic animals expressing heparanase, which can be used as a model for human disease and for the commercial production of heparanase. The present invention is further of compositions of matter produced by the transgenic animals and of methods of purifying heparanase therefrom. Specifically, the present invention can be used to produce commercial quantities of heparanase.

The principles and operation of the present invention may be better understood with reference to the drawings, examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention there is provided a transgenic animal expressing heparanase from a transgene.

As used herein the term “animal” refers to all multicellular organisms other than human.

As used herein the term “transgene” refers to a genetic construct including a a polynucleotide encoding for a heparanase protein. Preferably, the construct further including an additional polynucleotide harboring at least one cis-acting element which regulate the expression of heparanase from the first polynucleotide. The cis-acting element(s) are typically located upstream to the coding sequence encoding heparanase. When prepared, such a construct may include additional polynucleotides designed for propagating the construct in bacteria, preferably such additional polynucleotides are removed from the construct prior to the use thereof for generating the transgenic animal.

The phrase “expressing heparanase from a transgene” refers to transcription of heparanase messenger RNA (mRNA) followed by translation thereof into a heparanase. Post translational modifications, including glycosylation, prolteolytic cleavage and the like may follow translation.

Heparanase catalytic activity is known to include animal endoglycosidase hydrolyzing activity which is specific for heparin or heparan sulfate proteoglycan substrates, as opposed to the activity of bacterial enzymes (heparinase I, II and III) which degrade heparin or heparan sulfate by means of β-elimination.

Genes encoding mammalian heparanases and the expression and purification thereof are described in length in U.S. Pat. No. 5,986,822; U.S. Pat. application Ser. Nos. 09/071,739; 09/071,618; 09/109,386; 09/258,892; and PCT applications US/17954, US99/09255 and US99/09256, all of which are incorporated herein by reference. Further details and references are provided in the Background section above. It will be appreciated by one ordinarily skilled in the art, and it is demonstrated in the above patent documents, that using the human heparanase gene sequence one can readily clone, express and purify recombinant heparanase of any other mammal. This sequence of events, i.e., cloning a gene of one species based on the sequence of the same gene from another species, proved successful in hundreds of previous cases, especially since the polymerase chain reaction (PCR) may be practiced therefor.

Thus, the term “heparanase” includes polypeptides encoded by a mammalian heparanase gene or a portion thereof, e.g., the portion encoding the mature processed heparanase. The term also includes all of the heparanase species described and discussed in U.S. Pat. application Ser. No. 09/260,038; and in PCT/US99/09256, both are incorporated herein by reference. These species of heparanase are cleavable into active forms via specific proteases.

The ability to incorporate specific genes into the genome of mammalian embryos has provided a useful in vivo system for the analysis of gene control and expression. The high efficiency transformation of cultured mammalian cells has been accomplished by direct microinjection of specific DNA sequences into the cell nucleus (59). Gordon, J. W. et al. (60) demonstrated that DNA could be microinjected into mouse embryos, and found in the resultant offspring. The basic procedure used to produce transgenic mice requires the recovery of fertilized eggs from the oviducts of newly mated female mice. DNA, which contains the gene desired to be transferred into the mouse, is microinjected into the male pronucleus of each fertilized egg. Microinjected eggs are then implanted into the oviducts of one-day pseudopregnant foster mothers and carried to term (61). Such microinjected genes frequently integrate into chromosomes, are retained throughout development and are transmitted to offspring as Mendelian traits (61, 62). Microinjected foreign genes have shown a tendency to be expressed in transgenic mice. Similarly, other mammalian and non-mammalian species (e.g., avian species) are transgenized using similar techniques.

Thus, a variety of transgenic animal species are presently used to produce recombinant proteins.

For mammals, the general approach is to target the expression of the desired protein to the mammary gland using regulatory elements derived from a milk protein gene and then collect and purify the product from milk of animals for the production of the recombinant enzyme. Transgenic cows (see, U.S. Pat. Nos. 6,080,912; 6,013,857), ewes (see, U.S. Pat. Nos. 5,756,687; 6,087,554), goats (see, U.S. Pat. No. 5,843,705) and pigs (6.030,833; 5,942,435) can be readily engineered to produce recombinant proteins in the milk. Protocols for generating transgenic mammals are provided in, for example, U.S. Pat. Nos. 6,118,045; 6,018,097; 6,015,938; 5,994,616; 5,965,789; 5,965,788; 5,959,171; 5,891,698; 5,880,327; 5,861,313; 5,859,307; 5,850,000; 5,849,997; 5,849,992; 5,831,141; 5,827,690; 5,824,287; 5,759,536; 5,756,687; 5,750,172; 5,716,817; 5,714,345; 5,705,732; 5,700,671; 5,654,182; 5,648,243; 5,639,440; 5,635,355; and 5,602,300, which are incorporated herein by reference.

The following proteins have been successfully expressed in milk: lysosomal proteins; collagen, EC-SOD; bacteriostatic proteins, insulin and many more.

Thus, according to another aspect of the present invention there is provided a method of manufacturing heparanase by (a) obtaining a transgenic mammal having mammary glands, the mammal expressing recombinant heparanase and secreting the heparanase into milk being produced by the mammary glands; milking the mammal so as to obtain milk containing heparanase; and purifying the heparanase from the milk.

In addition, recombinant heparanase may be produced in eggs of transgenic hens. The general approach in this case is to target the expression of the desired protein to the egg producing cells using regulatory elements derived from an egg protein gene and then use the egg content as a source of heparanase (e.g., collect and purify the product from eggs of animals for the production of the recombinant enzyme). Protocols for generating transgenic avians are provided in, for example, U.S. Pat. Nos. 6,080,912; 6,018,097.

Thus, according to yet another aspect of the present invention there is provided a method of manufacturing heparanase by obtaining a transgenic avian having egg producing cells, the avian expressing recombinant heparanase and secreting the heparanase into eggs being produced by the egg producing cells; collecting eggs laid by the avian so as to obtain eggs containing heparanase; and purifying the heparanase from the eggs.

Methods of purifying heparanase are described in, for example, U.S. patent application Ser. Nos. 09/071,618 and 09/260,038, which are incorporated herein by reference.

As is well known in the art, a transgenic animal may include a single locus or several loci harboring the transgene. Southern blot analysis using specific restriction endonucleases can be used to monitor the number of copies of a transgene, so as quantitative PCR. In a specific animal, each such loci may be homozygous or heterozygote. Careful breading with wild type animals can be used to obtain homozygote or heterozygote animals. In addition, a transgene can be passed from a first genetic background of a first mating strain of a species to another genetic background of a second mating strain of that species by carefully implemented, and well known, breeding protocols. Typically, 3-5 generations are required to do so, depending on the level of heterogeneity between the matting strains.

The expression of the heparanase transgene may be tissue specific, non-specific (all or most tissues), inducible or constitutive. To this end any one of a great repertoire of tissue specific, non-specific, inducible or constitutive promoters can be used. Tissue specific promoters include, but are not limited to, beta-lactoglobulin promoter (Genebank Accession No. X52581), mammary glands (Clark 1998) Rb promoter (Genebank Accession No. M86180), nervous system (Jiang et al. 2000), preproendothelin-1 promoter (Genebank Accession No. U07982), and cardiovascular system (Zaidi et al. 1999). Non Tissue-specific constitutive promoters include, but are not limited to, beta-actin promoter and cytomegalovirus promoter. Inducible promoters include, but are not limited to,TetO (tet operator) promoter which is induced by doxycycline and metallothionein promoter (Genebank Accession No. X00504). Metallothionein expression is normally low in most tissues. High expression can be induced by several inflammatory cytokines, protein kinase C activators, and stress agents including heavy metals (Mirault ME et al. Ann N Y Acad Sci 1994 November 17;738:104-15). Using information derived from EST libraries, one can identify tissue specific or non-specific mRNAs and readily clone the promoters responsible for their expression, which reside upstream to the coding sequence in the respective genome.

Tissue specific or constitutive expression can be used according to the present invention not only to produce commercial quantities of heparanase, as described above and exemplified in the examples section that follows, but also to generate animal models for a variety of human diseases and for other applications as is further delineated hereinafter.

Any one or more of several methods can be used to monitor the expression of a transgene. These include tissue specific Northern blot; tissue specific RT-PCR; in situ hybridization; immunohistochemistry; and protein activity assays. These methods are well known in the art and are described in detail in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989).

Thus, for example, a transgenic mice overexpressing heparanase provides a powerful tool for studying the role of heparanase in normal and pathological processes. Using specific promoter and regulatory sequences, expression of a transgene can be defined to specific time and location. Expression pattern may reflect a specific mode of protein administration. In animals, which express the transgene constitutively in all tissues, heparanase is provided chronically and systemically.

Constitutive expression by a specific organ/tissue reflects administration of heparanase chronically but locally while inducible expression reflects either systematic or local administration in an acute manner.

Several clinical conditions have been correlated with heparanase expression, metabolism of heparan sulfate and HS bound proteins.

Transgenic animals over-expressing the heparanase may provide a model system for testing the role of heparanase in such conditions. The role of heparanase in cancer can be studied by testing the susceptibility of the transgenic mice to tumor growth and metastasis. A more comprehensive tool can be generated by crossing the transgenic mice with known mouse models for cancer research (e.g., mammary cancer, Guy et al. Proc. Natl. Acad. Sci. USA 1992, 89:10578-82, prostate cancer, Tomoyuki Shirai et al. Mutation research 2000, 412:219-226). Similarly, induction of inflammation and autoimmune disorders in heparanase overexpressing mice will shed light on heparanase involvement in such conditions. The effect of heparanase expression on development of which involve heparan sulfate and HS bound growth factors can also be evaluated and may suggest possible uses for therapy using gene therapy or the recombinant enzyme. Such conditions, which can be induced in the transgenic animals include tissue repair (e.g., wound healing, bone repair and nerve regeneration) where heparanase is suggested to increase the availability of HS bound growth factors and facilitate cell proliferation and migration, as well as pathological processes, which develop as a result of insufficient blood supply (e.g., cerebral, cardiac and diabetic ulcer ischemia), where heparanase is suggested to induce neovascularization. Transgenic mice can also serve as a model for studying the effect of heparanase on bone metabolism, including osteoporosis, either age related or in response to ovariohysterectomy, glucocorticoid therapy and heparin therapy and on amyloidosis, such as Alzheimer disease or renal.

Constitutive overexpression of heparanase may provide essential information regarding life long effects such as chronic toxicity as reflected by life span and aging, and the effect of heparanase on fertility and reproduction considering the suggested role of heparanase in embryo implantation (63).

In addition transgenic animals provide a source for primary cells overexpressing heparanase, such as embryonic cells, bone marrow cells, bone marrow stromal cells, spermatogonia, keratinocytes and sex cells (spermatocytes and oocytes). Such cells can be isolated using protocols for cell isolation and/or enrichment which are well known in the art. Based on the observation described in the Background section above that heparanase increases cell extravasation, such cells can be transplanted for immunotherapy, cell and gene therapy. Similarly, transgenic organs can be used for xenotransplantation, skin and embryo implantation, whereas sex cells can be used for in vitro fertilization (oocytes) and artificial insemination (spermatocytes).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials, Methods and Results

Immunohistochemistry:

Micrometer sections were deparaffinized and rehydrated. Tissue was then denatured for 3 minutes in a microwave oven in citrate buffer (0.01 M, pH 6.0). Blocking steps included successive incubations in 0.2% glycine, 3% H₂O₂ in methanol and 5% goat serum. Sections were incubated with a monoclonal anti-human heparanase antibody HP-130 (see U.S. Pat. application Ser. No. 09/071,739) diluted in PBS, or with DMEM supplemented with 10% horse serum as control, diluted as above, followed by incubation with HRP conjugated goat anti mouse IgG+IgM antibody (Jackson). Color was developed using Zymed AEC substrate kit (Zymed) for 10 minutes, followed by counter stain with Mayer's hematoxylin.

Preparation of Dishes Coated with ECM:

Bovine corneal EC were cultured as described in U.S. Pat. No. 5,986,822 except that 5% dextran T-40 was included in the growth medium and the cells were maintained without addition of bFGF for 12 days. The subendothelial ECM was exposed by dissolving the cell layer with PBS containing 0.5% Triton X-100 and 20 mM NH₄OH, followed by four washed in PBS. The ECM remained intact, free of cellular debris and firmly attached to the entire area of the tissue culture dish. For preparation of sulfate-labeled ECM, corneal endothelial cells were cultured in the presence of Na₂[³⁵S]O₄ (Amersham) added (25 pCi/ml) one day and 5 days after seeding and the cultures were incubated with the label without medium change. Ten to twelve days after seeding, the cell monolayer was dissolved and the ECM exposed.

Heparanase Activity:

Degradation of sulfate labeled ECM by heparanase was determined as described in U.S. Pat. No. 5,986,822. Briefly, ECM was incubated (24 hours, 37° C., pH 6.2) with recombinant heparanase or hpa-transfected cells and sulfate labeled material released into the incubation medium was analyzed by gel filtration on a Sepharose 6B column. Intact HSPGs were eluted just after the void volume (Kav<0.2, peak I) and HS degradation fragments eluted with 0.5<Kav<0.8 (peak II).

Generation of Heparanase Transgenic Mice:

High level constitutive expression of heparanase was driven by chicken beta-actin promoter. The plasmid pCAGGS (64) was modified to contain a unique EcoRI site at position 1719. An XbaI-EcoRI 1.7 kb fragment, which contained the entire open reading frame of heparanase was cloned into the compatible sites of the vector.

Before injection, the plasmid pCAGGS-hpa was digested with SalI and PstI in order to isolate the expression cassette and eliminate bacterial DNA sequences. The resulting fragment contained the CMV-IE enhancer, chicken β-actin promoter and hpa cDNA followed by a rabbit b-globin poly adenylation site.

The DNA fragment containing the hpa expression cassette was injected into fertilized eggs, derived from C57BL x BalbC breed. The isolation of fertilized eggs, injection of DNA and transplantation of blastocytes were conducted by the Department of cell biochemistry—the transgenic unit at the Hadassah Medical School, Jerusalem according to a protocol adapted from Hogan et al. Manipulating the Mouse Embryo A Laboratory Manual, Cold Spring Harbor Laboratory Press, 1994.

Mice developed from the injected blastocytes were tested for the presence of the human hpa transgene in their genome. Genomic DNA was extracted from tail tips of the mice and the human hpa transgene sequence was amplified using human hpa specific PCR primers. To this end, tail fragments were incubated overnight at 55° C. in a lysis buffer (8 M urea, 0.2 M Tris-HCl, 0.4 M NaCl, 20 mM EDTA, 1% N-Laurylsarcosine, 10 μg/ml proteinase K). The dissolved tissue underwent phenol extraction and ethanol precipitation, to obtain a highly purified genomic DNA.

The integration of the human heparanase cDNA in the mouse genome was verified by PCR using two sets of primers. The first couple was designed to amplify the 5′ region of the transgene. It included a β-actin promoter specific primer (designated 5′-pCAGGs) 5′-ATAGGCAGCTGACCTGA-3′ (SEQ ID NO:1) and human hpa specific primer: (designated Hpl-300) 5′-TGACTTGAGATTGCCAGTAACTTC-3′ (SEQ ID NO:2). The second primers set was designed to amplify the 3′ region of the transgene. It included a human hpa specific primer (designated Hpu-830) 5′-CTGTCCAACTCAATGGTCTAACTC-3′ (SEQ ID NO:3), and a primer specific to the plasmid derived 3′-untranslated region (designated 3′pCAGGS) 5′-TCTAGAGCCTCTGCTAACCA-3′ (SEQ ID NO:4); PCR conditions were as follows: 2 minutes at 95° C. followed by 33 cycles of 15 seconds at 95° C., 1 minute at 58° C. and 1 minute at 72° C.

Four Go founder mice were obtained, harboring the human hpa cDNA in their genome as revealed by a PCR reaction specific for the human hpa cDNA. Founders were mated with C57B1 mice to create F1 mice and those were mated among themselves to create F2 mice. Homozygous F2 mice from each Go line were identified by Southern blot analysis and a quantitative PCR assay. Homozygousity was verified by mating with C57B1 mice, where all the pups were positive heterozygous. All founder transgenic mice were back crossed with C57BL mice in order to establish C57B1 transgenic mice with a pure genetic background.

Expression of Human Heparanase in Transgenic Mice:

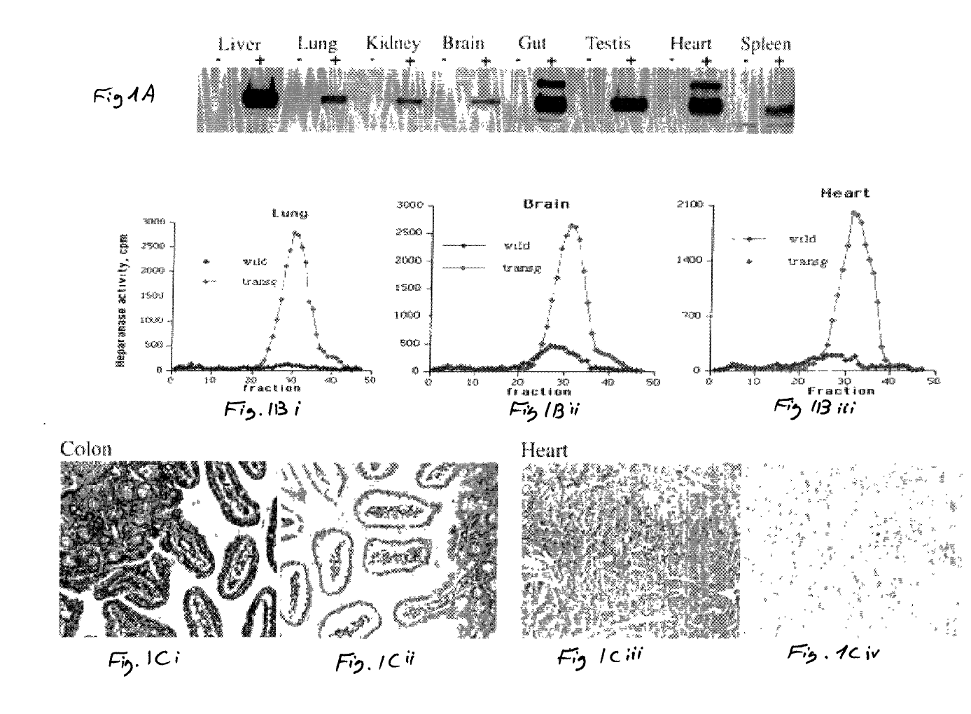

Expression of the heparanase protein was demonstrated by Western blot analysis of tissue extracts derived from F1 transgenic and control mice (FIG. 1A). Measurements of heparanase activity in tissue extracts revealed a much higher activity in the transgenic as compared to control mice in all tissues examined (FIGS. 1Bi-iii). Immunohistochemical staining of tissue sections revealed a high expression of the human heparanase protein in tissues derived from the transgenic mice but not control mice (FIGS. 1Ci-iv).

Phenotype of Human Heparanase Overexpressing Transgenic Mice:

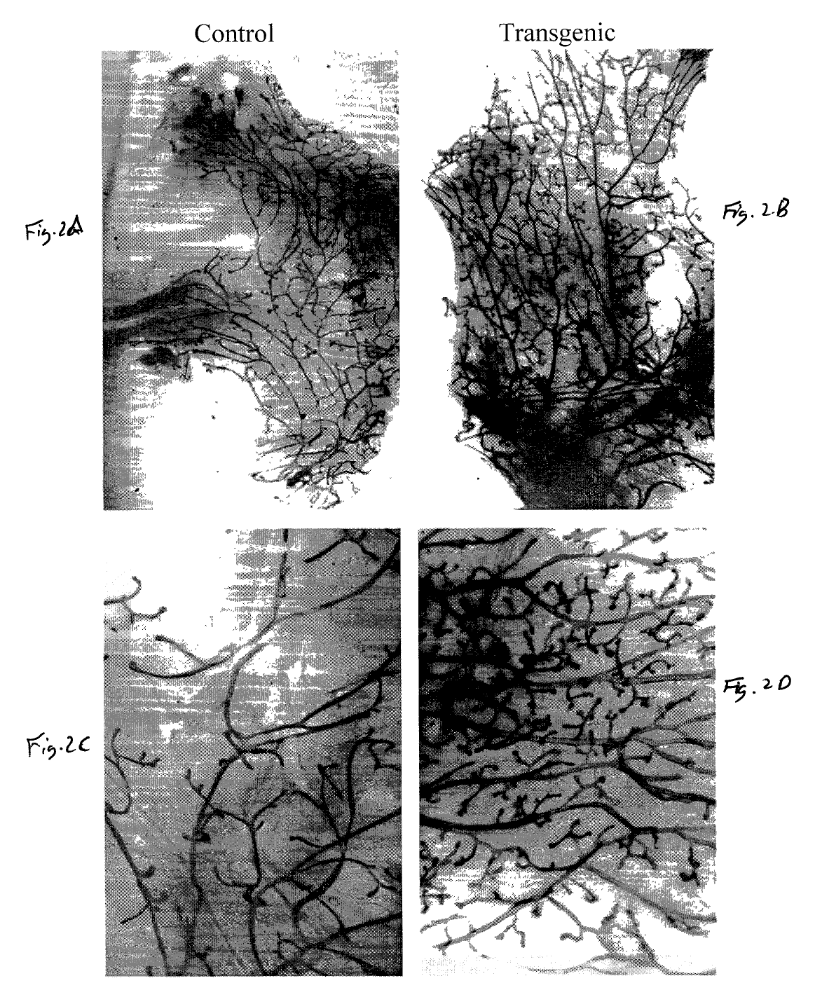

The transgenic mice are fertile and show no apparent signs of abnormality. Few phenotypic alterations were however noted. For example, the virgin transgenic mice develop lobular-alveoli structures in the mammary gland, a phenomenon that is characteristic of mammary glands of pregnant mice (FIGS. 2A-D).

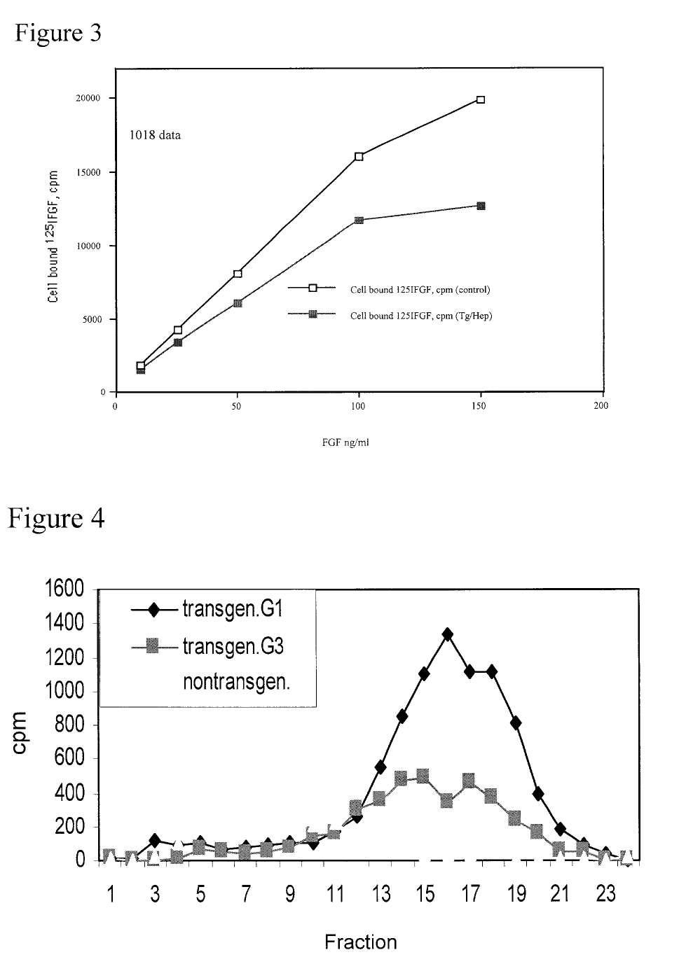

Overexpression of heparanase may lead to alterations in the amount and composition of heparan sulfate in the extracellular matrix (ECM) and surface of cells derived from the transgenic vs. control mice. In order to examine the effect of heparanase overexpression on cell surface heparan sulfate, the bFGF binding capacity of embryonic cells from transgenic and control mice was tested. Fibroblasts were isolated from embryos of transgenic mice and control mice 15 days post gestation. Cells were cultured in DMEM/RPMI/F-12 medium supplemented with 10% FCS. Confluent cells were incubated with various concentrations of radio-iodinated bFGF. Following incubation cells were washed and the bound bFGF was quantitated. As shown in FIG. 3, binding of bFGF to fibroblasts of transgenic embryos was lower than to fibroblasts of control embryos. This observation suggests that high levels of heparanase reduce the amount of heparan sulfate on the cell surface.

Heparanase in Milk of Transgenic Mice:

Milk of transgenic mice was tested for heparanase activity. Milk was obtained from females of two independent lines of transgenic mice and from control mice 7-10 days after delivery. Milk was diluted 1:10 in phosphate citrate buffer pH 6.0 and incubated on 35S labeled ECM for 48 hours. Degradation products were size fractionated. As shown in FIG. 4 heparanase activity was detected in the two transgenic lines G1 and G3, while no activity was detected in milk of control mice. This observation indicates that active heparanase can by produced in the mammary glands and secreted into the milk of transgenic animals.

Tissue Specific Expression of Heparanase in Transgenic Mice:

In more recent experiments, the hpa cDNA was cloned into a PES7 plasmid, a derivative of pSP72 containing the minimal apoAl promoter, driving expression of the human 7 alpha-hydroxylase enzyme exclusively in the liver of male mice. (PES7 expression vector was a gift from Schayek E., Bresbow L. B, The Rockefeller University NY. The 7 alpha-hydroxylase was replaced by the hpa cDNA in the proper orientation. Briefly, hpa cDNA was exiced from pCAGGS-hpa2 using XbaI. The 1.7 kb XbaI fragment was subcloned into the XbaI site of PES7 plasmid. The appropriate linear fragment was cut, purified and subjected to microinjection. A single transgenic mouse expressing the human hpa cDNA was obtained. This mouse was bred to produce F1 mice.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by a Genbank accession number mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

### REFERENCES CITED

1. Durand, P., Lehn, P., Callebaut, I., Fabrega, S., Henrissat, B. and Mornon, J. P. (1997). Active-site motifs of lysosomal acid hydrolases: invariant features of clan GH-A glycosyl hydrolases deduced from hydrophobic cluster analysis. Glycobiology, 7(2), 277-284.

2. Vlodavsky, I., Friedmann, Y., Elkin, M., Aingorn, H., Atzmon, R., Ishai-Michaeli, R., Bitan, M., Pappo, O., Peretz, T., Michal, I., Spector, L and Pecker I. (1999). Mammalian heparanase: gene cloning, expression and function in tumor progression and metastasis. Nature Medicine (In press)

3. Wight, T. N., Kinsella, M. G., and Qwamstromn, E. E. (1992). The role of proteoglycans in cell adhesion, migration and proliferation. Curr. Opin. Cell Biol., 4, 793-801.

4. Jackson, R. L., Busch, S. J., and Cardin, A. L. (1991). Glycosaminoglycans: Molecular properties, protein interactions and role in physiological processes. Physiol. Rev., 71, 481-539.

5. Wight, T. N. (1989). Cell biology of arterial proteoglycans. Arteriosclerosis, 9, 1-20.

6. Kjellen, L., and Lindahl, U. (1991). Proteoglycans: structures and interactions. Annu. Rev. Biochem., 60, 443-475.

7. Ruoslahti, E., and Yamaguchi, Y. (1991). Proteoglycans as modulators of growth factor activities. Cell, 64, 867-869.

8. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

9. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

10. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127.

11. Nakaji, M., Irimura, T., and Nicolson, G. L. (1988). Heparanase and tumor metastasis. J. Cell. Biochem., 36, 157-167.

12. Vlodavsky, I. et al. Invasion Metastasis 1995, 14:290-302.

13. Nakagima, M. et al J. Cell. Biochem. 1988, 36:157-167.

14. Vlodavsky, I. et al. Cancer res. 1983, 43:2704-2711.

15. Vlodavsky, I. et al. J. Med 1988, 24:464-470.

16. Vlodavsky, I. et al. Invasion and Metastasis 12:112-127.

17. Gilat, D. et al. J. Exp. Med. 1995, 181:1929-1934.

18. Matzner et al. 1985, J. Clin. Invest. 10:1306-1313.

19. Mollinedo, F. et al. Biochem. J. 1997, 327:917-923.

20. Murphy,G. et al. Biochem. J. 1990, 192:517-525.

21. Nakajima, M. et al. J. Cell. Biochem. 1988,36(2):157-167.

22. Ishai-Michaeli R. wt al. Cell Reg. 1990, 1:833-842.

23. Cardon-Cardo C. et al. Lab. Inrest. 1990, 63:832-840.

24. Nicolson, G. L. (1988). Organ specificity of tumor metastasis: Role of preferential adhesion, invasion and growth of malignant cells at specific secondary sites. Cancer Met. Rev., 7, 143-188.

25. Liotta, L. A., Rao, C. N., and Barsky, S. H. (1983). Tumor invasion and the extracellular matrix. Lab. Invest., 49, 639-649.

26. Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711.

27. Sun L, Feusi E, Sibalic A, Beck-Schimmer B, Wuthrich RP (1998). xpression profile of hyaluronidase mRNA transcripts in the kidney and in renal cells. Kidney Blood Press Res;21(6):413-8

28. Parish, C. R., Coombe, D. R., Jakobsen, K. B., and Underwood, P. A. (1987). Evidence that sulphated polysaccharides inhibit tumor metastasis by blocking tumor cell-derived heparanase. Int. J. Cancer, 40, 511-517.

29. Burgess, W. H., and Maciag, T. (1989). The heparin-binding (fibroblast) growth factor family of proteins. Annu. Rev. Biochem., 58, 575-606.

30. Folkman, J., and Klagsbrun, M. (1987). Angiogenic factors. Science, 235, 442-447.

31. Vlodavsky, I., Folkman, J., Sullivan, R., Fridman, R., Ishai-Michaelli, R., Sasse, J., and Klagsbrun, M. (1987). Endothelial cell-derived basic fibroblast growth factor: Synthesis and deposition into subendothelial extracellular matrix. Proc. Natl. Acad. Sci. USA, 84, 2292-2296.

32. Folkman, J., Klagsbrun, M., Sasse, J., Wadzinski, M., Ingber, D., and Vlodavsky, I. (1980). A heparin-binding angiogenic protein—basic fibroblast growth factor—is stored within basement membrane. Am. J. Pathol., 130, 393-400.

33. Bashkin, P., Doctrow, S., Klagsbrun, M., Svahn, C. M., Folkman, J., and Vlodavsky, I. (1989). Basic fibroblast growth factor binds to subendothelial extracellular matrix and is released by heparitinase and heparin-like molecules. Biochemistry, 28, 1737-1743.

34. Ishai-Michaeli, R., Svahn, C.-M., Chajek-Shaul, T., Korner, G., Ekre, H.-P., and Vlodavsky, I. (1992). Importance of size and sulfation of heparin in release of basic fibroblast factor from the vascular endothelium and extracellular matrix. Biochemistry, 31, 2080-2088.

35. Ishai-Michaeli, R., Eldor, A., and Vlodavsky, I. (1990). Heparanase activity expressed by platelets, neutrophils and lymphoma cells releases active fibroblast growth factor from extracellular matrix. Cell Reg., 1, 833-842.

36. Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271.

37. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp327-343. Academic press Inc., Orlando, Fla.

38. Yayon, A., Klagsbrun, M., Esko, J. D., Leder, P., and Ornitz, D. M. (1991). Cell surface, heparin-like molecules are required for binding of basic fibroblast growth factor to its high affinity receptor. Cell, 64, 841-848.

39. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible for FGF receptor dimerizatio, activation, and cell proliferation. Cell, 79, 1015-1024.

40. Ornitz, D. M., Herr, A. B., Nilsson, M., West, a., J., Svahn, C.-M., and Waksman, G. (1995). FGF binding and FGF receptor activation by synthetic heparan-derived di- and trisaccharides. Science, 268, 432-436.

41. Gitay-Goren, H., Soker, S., Vlodavsky, I., and Neufeld, G. (1992). Cell surface associated heparin-like molecules are required for the binding of vascular endothelial growth factor (VEGF) to its cell surface receptors. J. Biol. Chem., 267, 6093-6098.

42. Ernst S, Langer R, Cooney CL, Sasisekharan R. (1995). Enzymatic degradation of glycosaminoglycans. Crit Rev Biochem Mol Biol; 30(5):387-444

43. Current protocols in molecular biology (1994-1999). Ausubel, F. M., Brent, R., Kingston, R. E., Moore D. D., Seidman, J. G., Smith, J. A., Struhl, K. Eds. JohnWiley & Sons, Inc.

44. Rapraeger, A., Krufka, A., and Olwin, B. R. (1991). Requirement of heparan sulfate for bFGF-mediated fibroblast growth and myoblast differentiation. Science, 252, 1705-1708.

45. Shieh, M-T., Wundunn, D., Montgomery, R. I., Esko, J. D., and Spear, P. G. J. (1992). Cell surface receptors for herpes simplex virus are heparan sulfate proteoglycans. J Cell Biol., 116, 1273-1281.

46. Chen, Y., Maguire, T., Hileman, R. E., Fromm, J. R., Esko, J. D., Linhardt, R. J., and Marks, R. M. (1997). Dengue virus infectivity depends on envelope protein binding to target cell heparan sulfate. Nature Medicine 3, 866-871.

47. Putnak, J. R., Kanesa-Thasan, N., and Innis, B. L. (1997). A putative cellular receptor for dengue viruses. Nature Medicine 3, 828-829.

48. Narindrasorasak, S., Lowery, D., Gonzalez-DeWhitt, P., Poorman, R. A., Greenberg, B., Kisilevsky, R. (1991). High affinity interactions between the Alzheimer's beta-amyloid precursor protein and the basement membrane form of theparan sulfate proteoglycan. J. Biol. Chem., 266, 12878-83.

49. Eisenberg, S., Sehayek, E., Olivecrona, T., and Vlodavsky, I. (1992). Lipoprotein lipase enhances binding of lipoproteins to heparan sulfate on cell surfaces and extracellular matrix. J. Clin. Invest., 90, 2013-2021.

50. Ross, R. (1993). The pathogenesis of atherosclerosis: a perspective for the 1990s. Nature (Lond.)., 362:801-809.

51. Zhong-Sheng, J., Walter, J., Brecht, R., Miranda, D., Mahmood Hussain, M., Innerarity, T. L. and Mahley, W. R. (1993). Role of heparan sulfate proteoglycans in the binding and uptake of apolipoprotein E-enriched remnant lipoproteins by cultured cells. J. Biol. Chem., 268, 10160-10167.

52. Current protocols in protein science (1996-1999). Coligan, J. E., Dunn, B. M., Ploegh, H. L., Speicher, D. W. and Wingfield, P. T. Eds. John Wiley & Sons Inc.

53. Freeman C and Parish CR (1998) Human platelet heparanase: purification, characterization and catalytic activity.: Biochem J Mar 15;330 (Pt 3):1341-50.

54. Kussie et al. (1999) Biochem Biophys Res Commun. 261(1):183-7.

55. Hullet et al. (1999) Nature Medicine 5(7):803-809.

56. Toyoshima and Nakajima (1999). J. Biol. Chem. 274(34):24153-24160

57. Fairbanks et al. (1999) J. Biol. Chem. 274(42):29587-29590

58. Friedmann et al. (2000) Expression of heparanase in normal, dysplastic, and neoplastic human colonic mucosa and stroma : evidence for its role in colonic tumorigenesis. Am J Pathol. October 2000; 157(4):1167-75 59. Capecchi, M., Cell 22:479-488 (1980).

60. Gordon, J. W. et al. (Proc. Natl. Acad. Sci. USA 77:7380-7384 (1978)

61. Wagner, T. E. et al., Proc. Natl. Acad. Sci. USA 78:6376-6380 (1981)

62. Grosschedl. R. et al. Cell October 1984; 38(3):647-58

63. Dempsey, L. A. et al. Trends Biochem Sci. August 2000; 25(8):349-51

64. Niwa, H et al. Gene 108: 193-200, (1991)

65. Clark AJ, J Mammary Gland Biol Neoplasia July 1998; 3(3):337-50

66. Jiang et al. J Biol Chem 2001 Jan 5;276(1):593-600

67. Zaidi et al. J Clin Invest April 1999; 103 (8): 12 11-9

68. Mirault ME et al. Ann N YAcad Sci November 1994 17;73 8:104-15

69. Guy et al. Proc. Natl. Acad. Sci. USA 1992, 89:10578-82

70. Tomoyuki Shirai et al. Mutation research 2000, 412:219-226

71. Hogan et al. Manipulating the Mouse Embryo A Laboratory Manual, Cold Spring Harbor Laboratory Press, 1994

What is claimed is:

1. A transgenic animal expressing heparanase from a transgene.

2. The transgenic animal of claim 1, being homozygote for the transgene.

3. The transgenic animal of claim 1, being heterozygote for the transgene.

4. The transgenic animal of claim 1, having a single locus harboring the transgene.

5. The transgenic animal of claim 1, having at least two loci each harboring the transgene.

6. The transgenic animal of claim 1, wherein said heparanase is human heparanase.

7. The transgenic animal of claim 1, wherein said human heparanase is genetically modified to be cleavable into an active form via a protease.

8. The transgenic animal of claim 1, wherein said heparanase is processed by an endogenous protease of the animal into an active form.

9. The transgenic animal of claim 1, wherein said transgene encodes a processed and active form of heparanase.

10. The transgenic animal of claim 1, being a mammal.

11. The transgenic animal of claim 1, being an avian.

12. The transgenic animal of claim 1, wherein said heparanase is expressed under control of a tissue specific promoter.

13. The transgenic animal of claim 1, wherein said heparanase is expressed under control of a tissue non-specific promoter.

14. The transgenic animal of claim 1, wherein said heparanase is expressed under control of a constitutive promoter.

15. The transgenic animal of claim 1, wherein said heparanase is expressed under control of an inducible promoter.

16. The transgenic animal of claim 10, wherein said heparanase being expressed in and secreted by cells of a mammary glands of said mammal.

17. The transgenic animal of claim 11, wherein said heparanase being expressed in and secreted by egg producing cells of said avian.

18. Sex cells derived from the transgenic animal of claim 1.

19. Semen derived from the transgenic animal of claim 1.

20. An embryo derived from the transgenic animal of claim 1.

21. A composition of matter comprising milk containing heparanase.

22. A composition of matter comprising egg containing heparanase.

23. A method of manufacturing heparanase, the method comprising the steps of:
(a) obtaining a transgenic mammal having mammary glands, said mammal expressing recombinant heparanase and secreting said heparanase into milk being produced by said mammary glands;
(b) milking said mammal so as to obtain milk containing heparanase; and
(c) purifying said heparanase from said milk.

24. A method of manufacturing heparanase, the method comprising the steps of:
(a) obtaining a transgenic avian having egg producing cells, said mammal expressing recombinant heparanase and secreting said heparanase into eggs being produced by said egg producing cells;
(b) collecting eggs laid by said avian so as to obtain eggs containing heparanase; and
(c) purifying said heparanase from said eggs.


SEQUENCE LISTING (4 sequences)

SEQ ID NO: 1 - DNA, 17 bp, Artificial sequence
  Synthetic oligonucleotide
ataggcagctgacctga

SEQ ID NO: 2 - DNA, 24 bp, Artificial sequence
  Synthetic oligonucleotide
tgacttgagattgccagtaacttc

SEQ ID NO: 3 - DNA, 24 bp, Artificial sequence
  Synthetic oligonucleotide
ctgtccaactcaatggtctaactc

SEQ ID NO: 4 - DNA, 20 bp, Artificial sequence
  Synthetic oligonucleotide
tctagagcctctgctaacca